(19) United States
(12) Patent Application Publication
(10) Pub. No.: US 2012/0214821 A1
(43) Pub. Date: Aug. 23, 2012

(54) Lipidomic Biomarkers of Diabetes

(75) Inventors: Robert Gerszten, Brookline, MA (US); Thomas Wang, Lexington, MA (US); Eugene Rhee, Cambridge, MA (US); Clary Clish, Reading, MA (US)

(73) Assignees: THE BROAD INSTITUTE, INC., Cambridge, MA (US); THE GENERAL HOSPITAL CORPORATION, Boston, MA (US)

(21) Appl. No.: 13/355,110

(22) Filed: Jan. 20, 2012

Related U.S. Application Data
(60) Provisional application No. 61/435,049, filed on Jan. 21, 2011.

(51) Int. Cl. A61K 31/64; H01J 49/26; C09K 3/00; A61K 31/155; A61P 3/10

(52) U.S. Cl. 514/255.06; 514/635; 252/408.1; 250/282

(57) ABSTRACT

The invention, in some aspects, relates to methods for predicting a subject's risk of developing a glucose-related metabolic disorder, e.g., diabetes. In some aspects, the invention relates to methods for selecting and monitoring a treatment for a glucose-related metabolic disorder, e.g., diabetes.

### CLAIM OF PRIORITY

This application claims the benefit of U.S. Provisional Patent Application Ser. No. 61/435,049, filed on Jan. 21, 2011, the entire contents of which are hereby incorporated by reference.

### FEDERALLY SPONSORED RESEARCH OR DEVELOPMENT

This invention was made with Government support under Grant Nos. N01-HC-25195, R01-DK-HL081572, NIH DK81572, and T32-DK-00754023, awarded by the National Institutes of Health. The Government has certain rights in the invention.

### TECHNICAL FIELD

This invention relates to methods for using lipidomic biomarkers, e.g., specific triacylglycerols (TAGs), to determine risk of developing diabetes.

### BACKGROUND

Several prospective studies have identified dyslipidemia, particularly hypertriglyceridemia, as an independent predictor of incident type 2 diabetes mellitus (1-5). In contrast to a discrete metabolite such as glucose, however, plasma lipids are comprised of dozens of distinct molecules. For example, combinations of various acyl chains esterified to a glycerol backbone generate numerous unique triacylglycerols (TAGs). Standard clinical measurement of triacylglycerols relies on the measurement of total glycerol following acyl chain hydrolysis (6), thus obscuring this underlying diversity.

### SUMMARY

The present invention is based on the use of LC/MS-based profiling to identify a lipidomic signature of diabetes risk. This pattern is most notable among TAGs, and is at least in part attributable to the graded relationship between specific TAGs and insulin resistance. These findings, however, do not merely recapitulate available metrics of metabolic risk: combining the positive and negative risk information in select TAGs is able to identify individuals with a greater than 5 fold increased odds of future disease, above and beyond the information provided by age, sex, BMI, fasting glucose, fasting insulin, total triglycerides, and HDL cholesterol.

Thus, in one aspect, the invention provides methods for determining the risk of developing diabetes in a subject. The methods include detecting the presence and/or determining levels of one or more lipids in the sample, wherein the lipids are selected from the group consisting of TAG 52:1, TAG 50:0, PC 34:2, TAG 48:1, TAG 46:1, TAG 48:0, TAG 44:1, LPE 18:2, SM 22:0, PC 36:2, TAG 58:10, LPC 22:6, TAG 56:9, TAG 60:12, and PC 38:6. The presence of TAG 52:1, TAG 50:0, PC 34:2, TAG 48:1, TAG 46:1, TAG 48:0, TAG 44:1, LPE 18:2, SM 22:0, PC 36:2 (e.g., the presence of the biomarker above a threshold level), indicates an increased risk of developing diabetes in the subject, and the presence of TAG 58:10, LPC 22:6, TAG 56:9, TAG 60:12 2 (e.g., the presence of the biomarker above a threshold level), and PC 38:6 indicates a decreased risk of, or protection from, developing diabetes in the subject.

In some embodiments, the methods include determining levels of TAG 50:0 and TAG 58:10. In some embodiments, the methods include determining levels of TAG 50:0, TAG 58:10, and SM 22:0. In some embodiments, where the presence of levels of both a risk-associated lipid (i.e., TAG 52:1, TAG 50:0, PC 34:2, TAG 48:1, TAG 46:1, TAG 48:0, TAG 44:1, LPE 18:2, SM 22:0, PC 36:2) and a protection-associated lipid (i.e., TAG 58:10, LPC 22:6, TAG 56:9, TAG 60:12 2), indicates that the subject has neither increased risk nor protection from developing diabetes.

In some embodiments, the methods include the sample comprises serum or plasma from the subject.

In some embodiments, the methods further include selecting a treatment for the subject based on the lipids present in the sample. In some embodiments, the methods further include administering the selected treatment to the subject. In some embodiments, the treatment is administering to the subject an effective amount of at least one anti-diabetes compound.

In some embodiments, the subject has normal glucose tolerance. In some embodiments, the methods include the subject has at least one risk factor for diabetes. In some embodiments, the subjects have predominantly European ancestry.

In some embodiments, the levels of the lipids are determined using a mass spectrometer. In some embodiments, the levels are determined using GC-MS, LC-MS, or HPLC-MS.

In a further aspect, the invention provides kits for use in any of the methods described herein for determining the presence or risk of a glucose related metabolic disorder in a subject. The kits can include one or more control samples comprising predetermined levels TAG 52:1, TAG 50:0, PC 34:2, TAG 48:1, TAG 46:1, TAG 48:0, TAG 44:1, LPE 18:2, SM 22:0, PC 36:2, TAG 58:10, LPC 22:6, TAG 56:9, TAG 60:12, and PC 38:6; and instructions for use of the kit in a method for determining the presence or risk of a glucose related metabolic disorder described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Case-Control comparison for all lipid analytes in Framingham Heart Study (FHS). (A) Geometric mean ratio of each lipid analyte for cases versus controls in fasting pre-OGTT plasma. (B) Mean difference in percent change 2 hours after an oral glucose challenge in cases versus controls (% chg in cases minus % chg in controls). For both plots, each data point represents a distinct lipid analyte. (C) CVs for each lipid analyte across a total of 29 pooled plasma samples.

FIGS. 2A-B. Triacylglycerol pattern of diabetes risk in FHS. (A) Geometric mean ratio of TAG levels in cases versus controls in fasting pre-OGTT plasma. Each circle represents a distinct TAG, organized along the x-axis based on total acyl chain carbon number (left panel) or double bond content (right panel). The size of each circle is proportional to the standard deviation of the case/control ratios for each TAG; therefore, smaller circles indicate greater precision, whereas larger circles indicate lesser precision. Note, the two panels display the same data, simply arranged along the x-axis by a different variable. (B) Geometric mean ratio of TAG levels in the subset of cases and controls in the bottom quartile of HOMA-IR (mean HOMA-IR 1.03 for cases, 1.01 for controls, p=0.36), organized along the x-axis based on total acyl chain carbon number (left panel) or double bond content (right panel).

FIGS. 3A-E. Relationship between diabetes risk and acyl chain content in non-TAG lipid analytes. Geometric mean ratio of lipid levels in cases versus controls in fasting pre-OGTT plasma for (A) cholesterol esters (CEs), (B) lysophosphatidylcholines (LPCs), (C) phosphatidylcholines (PCs), (D) lysophosphatidylethanolamines (LPEs), and (E) sphingomyelins (SMs). Each data point represents a distinct lipid analyte, organized along the x-axis based on total acyl chain carbon number (left panel) or double bond content (right panel). The size of each circle is proportional to the standard deviation of the case/control ratios for each lipid; therefore, smaller circles indicate greater precision, whereas larger circles indicate lesser precision. Note, the two panels display the same data points, simply arranged along the x-axis by a different variable.

FIG. 4A. Triacylglycerol diabetes risk pattern following multivariable adjustment. Conditional logistic regression models were fitted to assess the association between baseline TAG levels and future diabetes, adjusting for age, sex, BMI, fasting glucose, fasting insulin, total triglycerides, and HDL cholesterol. The Odds ratio (OR) for future diabetes per standard deviation (SD) increment of TAG level is plotted for each TAG, organized along the x-axis based on total acyl chain carbon number (left panel) or double bond content (right panel). Solid circles, p<0.05 for relating diabetes to TAG.

FIGS. 4B-D. Significant TAG predictors in FHS. Box and whisker plots for clinical laboratory measures (B), TAGs associated with increased risk of type 2 diabetes (C), and TAGs associated with decreased risk of type 2 diabetes in FHS (D). The lines in the boxes indicate median levels; the lower and upper boundaries of the box represent the 25th and 75th percentiles respectively; the lower and upper whiskers represent the 5th and 95th percentiles respectively. The diamonds in the boxes indicate mean areas, and the corresponding percent difference between these means (case versus control) is shown in each figure.

FIGS. 4E-I. The downsloping relationship between diabetes risk and carbon number and double bond content persisted after multivariable adjustment for LPCs, PCs, and possibly LPEs, but not for CEs.

FIGS. 5A-D. Triacylglycerols and insulin action in FHS. (A) Mean percent change of each TAG with OGTT. (B) Mean percent change of each TAG with OGTT for individuals in the lowest (solid diamonds) and highest (hollow diamonds) quartiles of HOMA-IR. (C) Spearman correlation coefficient for each TAG with HOMA-IR. For (A-C), each data point represents a distinct TAG, organized along the x-axis based on total acyl chain carbon number (left panel) or double bond content (right panel). (D) For TAGs, the risk of diabetes following multivariable adjustment and correlation with HOMA-IR.

FIGS. 6A-E. Triacylglycerol responses to pharmacologic and physiologic perturbations in alternative cohorts. Mean percent change of each TAG (A) 60 minutes and (B) 120 minutes following glipizide administration in 20 non-diabetic individuals. (C) Mean percent change of each TAG following 4 doses of metformin in 20 non-diabetic individuals. (D) Geometric mean ratio of TAG levels in 10 individuals with type 2 diabetes versus 40 non-diabetic controls. (E) Mean percent change of each TAG following exercise treadmill testing in 50 individuals. For (A-E), each data point represents a distinct TAG, organized along the x-axis based on total acyl chain carbon number (left panel) or double bond content (right panel).

FIG. 7. Fatty acyl chain constituents of diabetes risk predictors. Individual fatty acids are listed in the middle. Lipid analytes associated with an increased risk of diabetes following multivariable adjustment (except SM 22:0) are listed on the left, and lipid analytes associated with a decreased risk of diabetes following multivariable adjustment are listed on the right. Lines connect individual lipids with their fatty acid constituents.

### DETAILED DESCRIPTION

The present inventors have developed a liquid chromatography/mass spectrometry (LC/MS)-based lipid profiling platform that measures intact lipids across a variety of lipid classes: triacylglycerols (TAGs), cholesterol esters (CEs), lysophosphatidylcholines (LPCs), phosphatidylcholines (PCs), lysophosphatidylethanolamines (LPEs), diacylglycerols (DAGs), and sphingomyelins (SMs). Within each lipid class, this method further distinguishes analytes on the basis of total acyl-chain carbon number and double bond content. These factors define each lipid's molecular weight, which in turn determines the lipid's detection in the mass spectrometer. This platform has been applied to the study of human plasma, and is able to reproducibly detect and quantify >100 lipid analytes in 10 μL of starting volume. Because TAGs are composed of 3 acyl chains, this class of lipids has a particularly broad range of molecular weights; in one embodiment, the platform monitors 42 distinct TAGs. Discriminating plasma lipids at this level of detail has the potential to improve diabetes prediction, and shed insight on the intersection between dyslipidemia and metabolic risk.

Current technologies enable high-throughput ‘snapshots’ of the lipidome (13-15). In some embodiments, the present methods can include the use of LC/MS-based lipid profiling to identify a plasma signature of diabetes risk. As described herein, TAGs of lower carbon number and double bond content are associated with an increased risk of type 2 diabetes, whereas TAGs of higher carbon number and double bond content are associated with a decreased risk of type 2 diabetes. A similar pattern holds for other lipid classes, including LPCs, LPEs, and PCs. Without wishing to be bound by theory, the results of physiologic and pharmacologic experiments suggest that the divergent risk embedded in plasma triglycerides is due in part to the heterogeneous relationship between individual TAGs and insulin action. Nevertheless, select TAGs and other lipid analytes remain significant disease predictors after adjusting for insulin (as well as other biochemical and clinical risk factors), and among the subset of subjects in the lowest quartile of HOMA-IR.

Several lines of evidence demonstrate that lipid profiling helps clarify the relationship between plasma TAGs and insulin action. As described herein, in the acute setting, TAGs of lower carbon number and double bond content decrease with OGTT, whereas TAGs of relatively higher carbon number and double bond content increase. These findings were not appreciated during recent metabolomic surveys of oral glucose ingestion (9, 16, 17). Glipizide administration results in the same dynamic TAG pattern, highlighting insulin rather than glucose as the proximate cause of the observed changes. The inverse pattern is elicited by acute metformin intake, which decreases plasma glucose and insulin levels. Exercise, which is known to acutely improve insulin sensitivity at the tissue level (11, 12), demonstrates the same TAG response as OGTT and glipizide administration. In a small study of 19 individuals, Schwab et at have shown that the sustained increase in insulin sensitivity associated with diet induced weight loss over 33 weeks is also associated with this pattern of TAG changes (18).

These observations are further corroborated by the relationship between plasma TAGs and insulin resistance. In fasting pre-OGTT FHS samples, TAGs of lower carbon number and double bond content—e.g., TAGs that fall in response to insulin action—are elevated in the setting of insulin resistance. Further, insulin resistant individuals have a blunted decrease in these TAGs during OGTT. TAGs of higher carbon number and double bond content, which increase in response to insulin action, have the weakest correlation with insulin resistance. Thus, individual TAGs respond differentially to insulin activity and sensitivity, both acutely and over time.

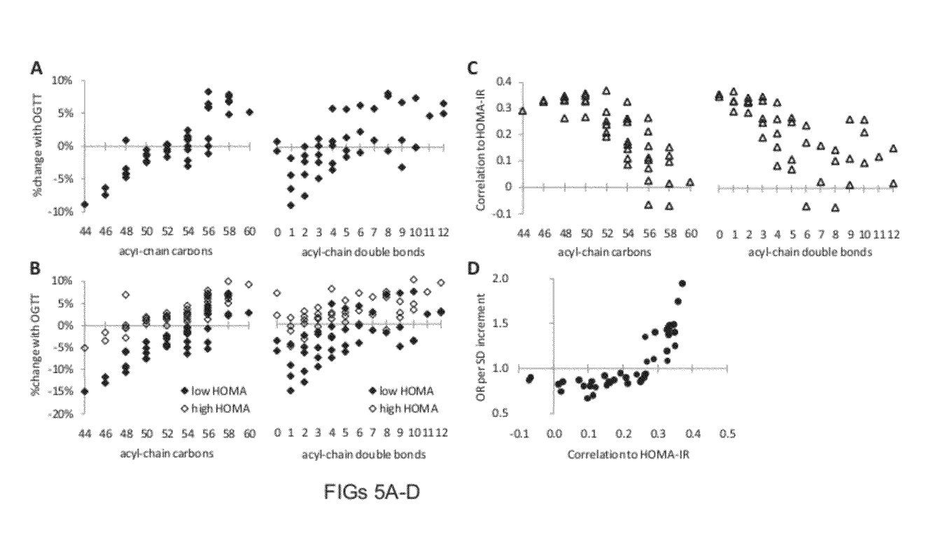

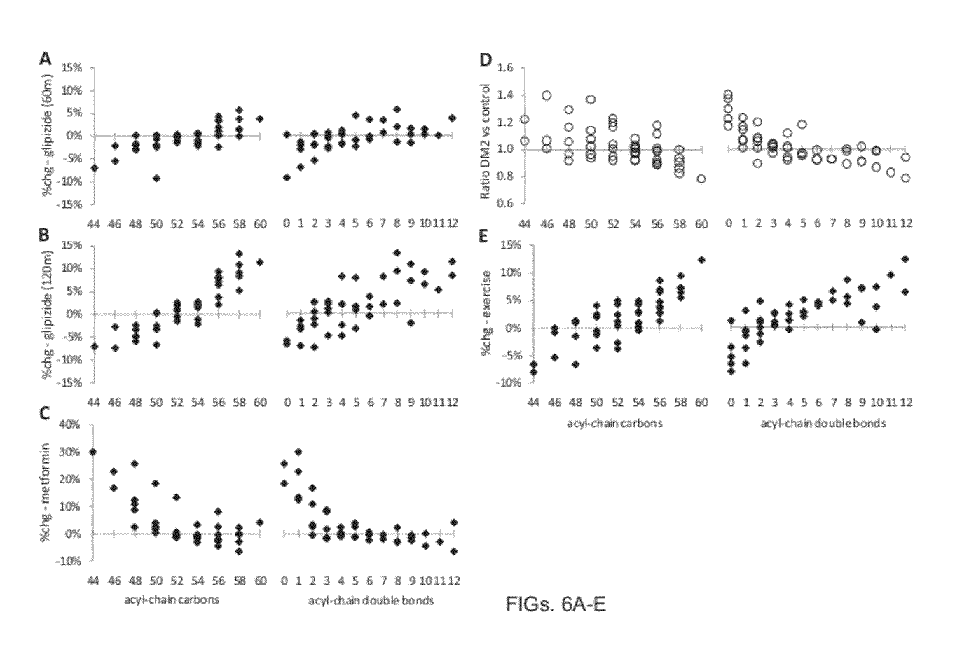

The results described herein demonstrate a positive relationship between each TAG's correlation with insulin resistance and its ability to predict type 2 diabetes in FHS (FIG. 5D). Contrary to the prevailing view of bulk triglycerides as an adverse risk factor, the present studies have identified specific TAGs that are associated with either an increased or decreased risk of diabetes. Further, these risk markers are altered up to 12 years prior to disease onset. The relative risks associated with these analytes are quite large in the population studied, and are comparable or higher than those associated with SD increments in age, fasting glucose, or BMI in prior population-based studies (19). Integrating the positive and negative risk captured by a TAG of relatively lower carbon number and double bond content (TAG 50:0) and a TAG of relatively higher carbon number and double bond content (TAG 58:10) further improves diabetes prediction. Finally, lipid profiling applied to individuals with and without type 2 diabetes demonstrates that the TAG risk pattern identified in FHS persists in established disease (FIG. 6C).

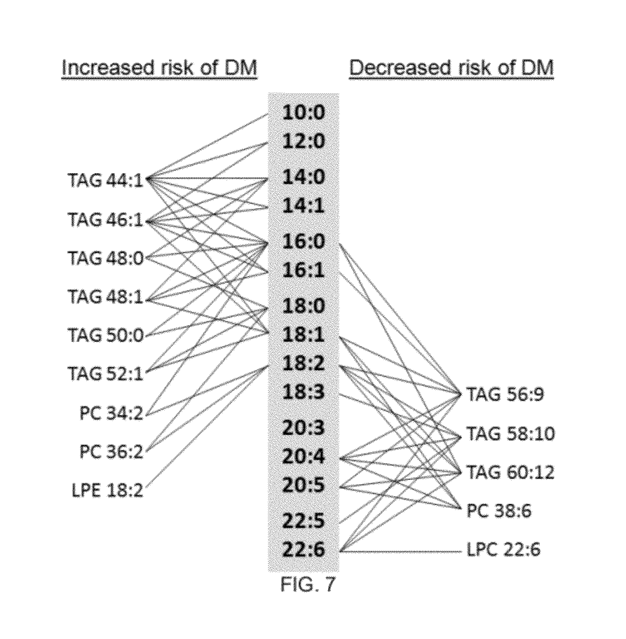

The results of MS/MS analyses demonstrate that the lipid analytes associated with increased diabetes risk are predominantly comprised of saturated and monounsaturated fatty acids, whereas lipids associated with decreased diabetes risk are comprised of polyunsaturated fatty acids (FIG. 7). These data are consistent with prior studies of diabetes prediction, which have relied on the measurement of derivatized fatty acids following hydrolysis of plasma lipids (20-23). By contrast, the present approach is able to view acyl chains in their natural context, across distinct macromolecular species. For instance, dynamic changes following glucose ingestion were notable among TAGs, but not SMs, PCs, or CEs. This finding directs attention towards TAG-specific mechanisms of acute insulin action. As an example, the increasing proportion of polyunsaturated fatty acids in TAGs during OGTT has been attributed to insulin mediated inhibition of hormone sensitive lipase: the subsequent decrease in saturated and monounsaturated free fatty acid release from adipose tissue increases the relative amount of polyunsaturated free fatty acids available to the liver for TAG assembly (17, 24). In contrast to the TAG-predominant response to OGTT, the relationship between diabetes risk and acyl chain composition in fasting pre-OGTT plasma was identified across several lipid classes. The breadth of this finding draws attention to general pathways of lipoprotein assembly. For example, insulin is known to increase the hepatic expression of various fatty acid desaturases, including SCD1, D5D, and D6D (25-28), in animals. Whether decreased desaturase activity due to insulin resistance contributes to the lipid risk pattern observed in humans remains unclear.

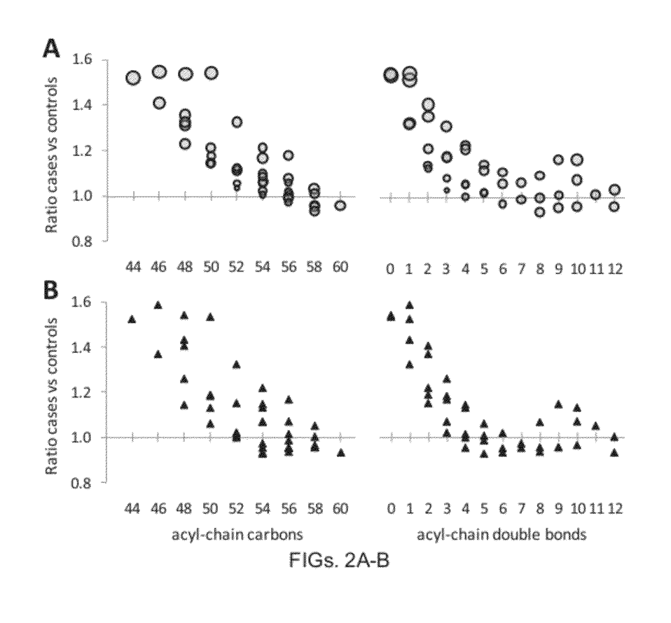

Although the upstream significance of insulin action has been highlighted, the conditional logistic regression model used in one embodiment described herein adjusted for baseline differences in fasting insulin, as well as age, sex, BMI, fasting glucose, total triglycerides, and HDL cholesterol. Further, the downsloping TAG risk pattern persists in the comparison between cases and controls in the lowest quartile of HOMA-IR (FIG. 2B). Other significant disease predictors such as SM 22:0 demonstrate no correlation with HOMA-IR, and improve risk prediction when combined with TAGs. Finally, dietary differences as culled from a detailed questionnaire do not account for differences in lipid profiles between cases and controls. These findings raise the possibility that select lipid predictors not only convey very subtle metabolic disturbances, but could also play a causal role in disease pathogenesis.

The present methods do not require comprehensive coverage of the plasma lipidome, but rather focus on abundant plasma lipids, allowing the measurement of >100 analytes in 10 μL samples; this feature may facilitate its clinical implementation. Kotronen et al. (29) have shown that lipid profiling of distinct lipoprotein fractions can also inform the relationship between individual lipids and insulin resistance; such an approach can provide valuable biologic insights, lipoprotein fractionation is impractical for high throughput biomarker applications. The present methods do not require absolute quantitation of lipid analytes, allowing for accurate detection of a pattern of diabetes risk, and the effect of insulin action on this pattern, without necessitating the absolute quantitation of any specific analyte. However, in cases where absolute quantitation is desired, methods known in the art such as incorporation of isotope labeled standards can be used for absolute quantitation of analytes.

Glucose-Related Metabolic Disorders

The invention, in some aspects, relates to methods, compositions and kits useful for diagnosing and determining risk of developing glucose-related metabolic disorders. As used herein, “glucose-related metabolic disorders” refer broadly to any disorder, disease, or syndrome characterized by a deficiency in the regulation of glucose homeostasis (e.g., hyperglycemia). Typically a glucose-related metabolic disorder is associated with abnormal insulin levels, insulin activity, and/or sensitivity to insulin (e.g., insulin resistance). As used herein diabetes (also referred to as diabetes mellitus), refers to any one of a number of exemplary classes (or types) of glucose-related metabolic disorders. Diabetes includes, but is not limited to the following classes (or types): type I diabetes mellitus, type II diabetes mellitus, gestational diabetes, and other specific types of diabetes. Glucose-related metabolic disorders also include prediabetic conditions, such as those associated with impaired fasting glycemia and impaired glucose tolerance. Glucose-related metabolic disorders are often associated with symptoms in a subject such as increased thirst and urine volume, recurrent infections, unexplained weight loss and, in severe cases, drowsiness and coma; high levels of glycosuria are often present. Children suspected of having a glucose-related metabolic disorder may, in some cases, present with severe symptoms, such as high blood glucose levels, glycosuria, and/or ketonuria.

Type 1 diabetes is usually due to autoimmune destruction of the pancreatic beta cells. Type 2 diabetes is characterized by insulin resistance in target tissues, which may result in a need for abnormally high amounts of insulin and diabetes develops when the beta cells cannot meet this demand. Gestational diabetes is similar to type 2 diabetes in that it involves insulin resistance; the hormones of pregnancy can cause insulin resistance in women genetically predisposed to developing this condition. Other specific types of diabetes are known in the art and disclosed in Definition, Diagnosis and Classification of Diabetes Mellitus and its Complications, Report: WHO/NCD/NCS/99.2 by the World Health Organisation, Department of Noncommunicable Disease Surveillance (1999), the contents of which are incorporated herein in their entirety by reference.

In some embodiments, the glucose-related metabolic disorder is Type 2 diabetes. Type 2 is also referred to as non-insulin-dependent diabetes or adult-onset diabetes, and is characterized by disorders of insulin action and insulin secretion, either of which may be the predominant feature. Both are usually present at the time that this form of diabetes is clinically manifest.

In some embodiments, the glucose-related metabolic disorder is gestational hyperglycemia or gestational diabetes. These are forms of diabetes associated with pregnancy. Gestational diabetes is associated with carbohydrate intolerance resulting in hyperglycemia of variable severity with onset or first recognition during pregnancy. Thus, it does not exclude the possibility that the glucose intolerance may antedate the pregnancy but was previously unrecognized. The classification typically applies irrespective of whether or not insulin is used for treatment or the condition persists after pregnancy.

In some embodiments, the glucose-related metabolic disorder is “Metabolic Syndrome” which is often characterized by hypertension, central (upper body) obesity, and dyslipidaemia, with or without hyperglycaemia. Subjects with the Metabolic Syndrome are at high risk of macrovascular disease. Often a person with abnormal glucose tolerance will be found to have at least one or more of the other cardiovascular disease (CVD) risk components. The Metabolic Syndrome is also referred to as Syndrome X and the Insulin Resistance Syndrome. Epidemiological studies confirm that this syndrome occurs commonly in a wide variety of ethnic groups including Caucasians, African-Americans, Mexican-Americans, Asian Indians, Chinese, Australian Aborigines, Polynesians and Micronesians. The Metabolic Syndrome with normal glucose tolerance identifies a subject as a member of a group at very high risk of diabetes. Thus, vigorous early management of the syndrome may have a significant impact on the prevention of both diabetes-and cardiovascular disease.

Diagnosis/Characterization

The present invention relates to methods useful for the characterization (e.g., clinical evaluation, diagnosis, classification, prediction, profiling) of glucose-related metabolic disorders, such as diabetes, based on the levels, presence, or absence of certain lipids referred to herein as biomarkers, or lipidomic biomarkers. As used herein, levels refer to the amount or concentration of a lipid or class of lipids in a sample (e.g., a plasma sample) or subject. The level may be expressed as an exact quantity, or may be expressed as a ratio to a reference lipid. In some embodiments, the methods include simply detecting the presence or absence of a specific lipid or type of lipid in a sample. In some cases, the methods can include determining whether a lipid is present in a concentration or a ratio above or below a reference level or ratio.

In some embodiments, the methods involve determining the ratio or levels of one or a plurality of lipiodomic biomarkers in a clinical sample, comparing the result to a reference, and characterizing (e.g., diagnosing, classifying) the sample based on the results of the comparison. A clinical sample can be any biological specimen (e.g., a blood sample) useful for characterizing the glucose-related lipidomic disorder (e.g., diabetes). Typically, a clinical sample contains one or more lipids. Exemplary biological specimens can include blood, serum, or plasma. In preferred embodiments, a clinical sample is a plasma sample.

In some embodiments, clinical samples are obtained from subjects (also referred to herein as individuals). As used herein, a subject is a mammal, including but not limited to a dog, cat, horse, cow, pig, sheep, goat, chicken, rodent, or primate (e.g., a human). Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. In some embodiments, a subject is a diabetic animal model. Diabetes animal models are well known in the art, for example: Leiter, Curr Protoc Immunol. 2001 May; Chapter 15: Unit 15.9; Levine et al., Am J Physiol Regul Integr Comp Physiol. 2008 Apr. 16; Oh Y S, et al., Diabetologia. 2008 Apr. 12; Sasaki et al., Arterioscler Thromb Vasc Biol. 2008 Apr. 10; Beauquis et al., Exp Neurol. 2008 April; 210(2):359-67; Cheng et al., Mol. Pharm. 2008 January-February; 5(1):77-91; Tikellis et al., Atherosclerosis. 2007 Dec. 17; Novelli et al., Pancreas. 2007 November; 35(4): e10-7; and Khazaei et al., Physiol Res. 2007 Nov. 30. Preferred subjects are humans (human subjects). The human subject may be a pediatric or adult subject. In some embodiments the adult subject is an overweight (BMI of 25-29) or obese (BMI of 30 or higher) subject.

In some embodiments, the methods involve diagnosing glucose-related metabolic disorder in a subject. To practice the diagnostic methods the levels of a plurality of biomarkers are typically determined. These levels are compared to a reference wherein the levels of the plurality of biomarkers in comparison to the reference is indicative of whether or not the subject has a glucose related metabolic disorder and/or should be diagnosed with the glucose related metabolic disorder.

As used herein, diagnosing includes both diagnosing and aiding in diagnosing. Thus, other diagnostic criteria may be evaluated in conjunction with the results of the methods herein in order to make a diagnosis.

The methods described herein are also useful for assessing the likelihood (or risk) of, or aiding in assessing the likelihood (or risk) of, a subject having or developing a glucose-related metabolic disorder. To practice the methods levels of a plurality of biomarkers are typically determined. These levels are compared to a reference wherein the levels or ratios of the plurality of biomarkers in comparison to the reference levels or or ratios is indicative of the likelihood that the subject will develop a glucose related metabolic disorder.

Other criteria for assessing likelihood that are known in the art (e.g., Body Mass Index (BMI), family history) can also be evaluated in conjunction with the methods described herein in order to make a complete likelihood assessment.

In some embodiments, methods involve determining the glucose control capacity or insulin sensitivity of a subject. To practice the methods, typically the levels of a plurality of biomarkers are determined. These levels are compared to a reference wherein the levels of the plurality of biomarkers in comparison to the reference are indicative of the glucose control capacity or insulin sensitivity.

As used herein, insulin sensitivity refers to the responsiveness of a subject, or cells of a subject, to the effects of insulin. For example, subjects with insulin resistance are less sensitive to insulin and therefore, have low insulin sensitivity. Techniques for measuring insulin sensitivity are well known in the art and include, for example, the hyperinsulinemic euglycemic clamp (i.e., the “clamp” technique), the Modified Insulin Suppression Test, fasting insulin levels, and glucose tolerance tests (e.g., an Oral Glucose Tolerance Test). The methods disclosed herein are also useful to characterize and obtain further insight on insulin sensitivity.

As used herein, glucose control capacity refers to a subject's ability (capacity) to control glucose levels within homeostatic limits (a physiologically safe/normal range). Consequently, insulin (and therefore insulin sensitivity), among other things, influences a subject's glucose control capacity. Other regulatory factors (e.g., hormones) in addition to insulin, such as glucagon, that influence glucose control capacity of a subject are well known in art. The methods disclosed herein are useful to characterize and obtain further insight on glucose control capacity.

The levels of the lipids for a subject can be obtained by any art recognized method. Typically, the level is determined by measuring the level of the lipid in a sample comprising plasma, or serum. The level can be determined by any method known in the art, e.g., ELISA, immunoassays, enzymatic assays, spectrophotometry, colorimetry, fluorometry, bacterial assays, liquid chromatography, gas chromatography, mass spectrometry, gas chromatography-mass spectrometry (GC-MS), liquid chromatography-mass spectrometry (LC-MS), LC-MS/MS, tandem MS, high pressure liquid chromatography (HPLC), HPLC-MS, and nuclear magnetic resonance spectroscopy, or other known techniques for determining the presence and/or quantity of a lipid; in some embodiments, the level is determined using one of LC-MS, HPLC-MS, or GC-MS. See, e.g., Suhre et al., Metabolic Footprint of Diabetes: A Multiplatform Metabolomics Study in an Epidemiological Setting. PLoS ONE 5(11): e13953 (2010). Conventional methods include sending a clinical sample(s) to a clinical laboratory, e.g., on site or a third party contractor, e.g., a commercial laboratory, for measurement.

In some cases, the methods disclosed herein involve comparing levels or occurrences (e.g., presence or absence) to a reference. The reference can take on a variety of forms. In some cases, the reference comprises predetermined values for a plurality of lipids (e.g., each of the plurality of lipids). The predetermined value can take a variety of forms. It can be a level or occurrence of a lipid in a control subject (e.g., a subject with a glucose-related metabolic disorder (i.e., an affected subject) or a subject without such a disorder (i.e., a normal subject)). It can be a level or occurrence of a lipid in a fasting subject. It can be a level or occurrence in the same subject, e.g., at a different time point. A predetermined value that represents a level(s) of a lipid is referred to herein as a predetermined level. A predetermined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

Subjects associated with predetermined values are typically referred to as control subjects (or controls). A control subject may or may not have a glucose related metabolic disorder (e.g., diabetes). In some cases it may be desirable that control subject is a diabetic, and in other cases it may be desirable that a control subject is a non-diabetic. Thus, in some cases the level of a lipid in a subject being greater than or equal to the level of the lipid in a control subject is indicative of a clinical status (e.g., indicative of a glucose-related metabolic disorder diagnosis). In other cases the level of a lipid in a subject being less than or equal to the level of the lipid in a control subject is indicative of a clinical status. The amount of the greater than and the amount of the less than is usually of a sufficient magnitude to, for example, facilitate distinguishing a subject from a control subject using the disclosed methods. Typically, the greater than, or the less than, that is sufficient to distinguish a subject from a control subject is a statistically significant greater than, or a statistically significant less than. In cases where the level of a lipid in a subject being equal to the level of the lipid in a control subject is indicative of a clinical status, the “being equal” refers to being approximately equal (e.g., not statistically different).

The predetermined value can depend upon a particular population of subjects (e.g., human subjects) selected. For example, an apparently healthy population will have a different ‘normal’ range of lipids than will a population of subjects which have, or are likely to have, a glucose-related metabolic disorder. Accordingly, the predetermined values selected may take into account the category (e.g., healthy, at risk, diseased) in which a subject (e.g., human subject) falls. Appropriate ranges and categories can be selected with no more than routine experimentation by those of ordinary skill in the art.

In some cases a predetermined value of a lipidomic biomarker is a value that is the average for a population of healthy subjects (human subjects) (e.g., human subjects who have no apparent signs and symptoms of a glucose-related metabolic disorder). The predetermined value will depend, of course, on the particular lipid (biomarker) selected and even upon the characteristics of the population in which the subject lies. In characterizing likelihood, or risk, numerous predetermined values can be established.

A level, in some embodiments, may itself be a relative level that reflects a comparison of levels between two states. For example, a level may be a relative level that reflects a comparison between fasting (e.g., pre-glucose consumption) and non-fasting states (e.g., post-glucose consumption). Where levels are relative levels that reflect a comparison between fasting and non-fasting states, the non-fasting state may be, for example, about 30 minutes, about 60 minutes, about 90 minutes, about 120 minutes, or more, post glucose consumption. In some cases, relative levels may be determined (e.g., by clinical personnel) during a standard oral glucose tolerance test, e.g., a first or baseline level that is obtained before the test and a second level that is obtained after the glucose consumption). Relative levels that reflect a comparison (e.g., ratio, difference, logarithmic difference, percentage change, etc.) between two states (e.g., fasting and non-fasting) may be referred to as delta values. For example, in the case of an oral glucose tolerance test, delta values may be a percentage change in levels of a biomarker from fasting to non-fasting states. The use of relative levels is beneficial in some cases because, to an extent, they exclude measurement related variations (e.g., laboratory personnel, laboratories, measurements devices, reagent lots/preparations, assay kits, etc.). However, the invention is not so limited.

Kits

The invention also provides kits for evaluating biomarkers in a subject. The kits of the invention can take on a variety of forms. Typically, the kits will include reagents suitable for determining levels of a plurality of biomarkers (e.g., those disclosed herein, for example as outlined in FIG. 7) in a sample. Optionally, the kits may contain one or more control samples or references. Typically, a comparison between the levels of the biomarkers in the subject and levels of the biomarkers in the control samples is indicative of a clinical status (e.g., diagnosis, likelihood assessment, insulin sensitivity, glucose control capacity, etc.). Also, the kits, in some cases, will include written information (indicia) providing a reference (e.g., predetermined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

Treatment

The present methods can also be used for selecting a treatment and/or determining a treatment plan for a subject, based on the occurrence or levels of certain lipids relevant to the glucose related metabolic disorders. In some embodiments, using the method disclosed herein, a health care provider (e.g., a physician) identifies a subject as having, or at risk of having or developing, a glucose-related metabolic disorder (e.g., Type II Diabetes) and, based on this identification the health care provider determines an adequate treatment plan for the subject. In some embodiments, using the method disclosed herein, a health care provider (e.g., a physician) diagnoses a subject as having, or at risk of having or developing, a glucose-related metabolic disorder (e.g., Type II Diabetes) based on the occurrence or levels of certain lipids in a clinical sample obtained from the subject, and/or based on a classification of a clinical sample obtained from the subject. By way of this diagnosis the health care provider determines an adequate treatment or treatment plan for the subject. In some embodiments, the methods further include administering the treatment to the subject.

In some embodiments, the invention relates to identifying subjects who are likely to have successful treatment with a particular drug dose, formulation and/or administration modality. Other embodiments include evaluating the efficacy of a drug using the metabolic profiling methods of the present invention. In some embodiments, the metabolic profiling methods are useful for identifying subjects who are likely to have successful treatment with a particular drug or therapeutic regiment. For example, during a study (e.g., a clinical study) of a drug or treatment, subjects who have a glucose-related metabolic disorder may respond well to the drug or treatment, and others may not. Disparity in treatment efficacy is associated with numerous variables, for example genetic variations among the subjects. In some embodiments, subjects in a population are stratified based on the metabolic profiling methods disclosed herein. In some embodiments, resulting strata are further evaluated based on various epidemiological, and or clinical factors (e.g., response to a specific treatment). In some embodiments, stratum, identified based on a metabolic profile, reflect a subpopulation of subjects that response predictably (e.g., have a predetermined response) to certain treatments. In further embodiments, samples are obtained from subjects who have been subjected to the drug being tested and who have a predetermined response to the treatment. In some cases, a reference can be established from all or a portion of the lipids from these samples, for example, to provide a reference metabolic profile. A sample to be tested can then be evaluated (e.g., using a prediction model) against the reference and classified on the basis of whether treatment would be successful or unsuccessful. A company and/or person testing a treatment (e.g., compound, drug, life-style change) could discern more accurate information regarding the types or subtypes of glucose-related metabolic disorders for which a treatment is most useful. This information also aids a healthcare provider in determining the best treatment plan for a subject.

In some embodiments, treatment for the glucose-related metabolic disorder is to administer to the subject an effective amount of at least one anti-diabetes compound and/or to instruct the subject to adopt at least one anti-diabetic lifestyle change. Anti-diabetes compound are well known in the art and some are disclosed herein. Non-limiting examples include alpha-glucosidase inhibitors for example acarbose and miglitol; biguanides for example metformin, phenformin, and buformin; meglitinides for example, repaglinide and nateglinide; sulfonylureas, for example tolbutamide, chlorpropamide, tolazamide, acetohexamide, glyburide, glipizide, glimepiride, and gliclazide; thiazolidinediones, for example troglitazone, rosiglitazone, and pioglitazone; peptide analogs, for example glucagon-like peptide I (GLP1) and analogs thereof (e.g., Exentide, Extendin-4, Liraglutide, gastric inhibitory peptide (GIP) and analogs thereof; vanadates (e.g., vanadyl sulfate); GLP agonists; DPP-4 inhibitors, for example vildagliptin and sitagliptin; dichloroacetic acid; amylin; carnitine palmitoyltransferase inhibitors; B3 adrenoceptor agonists; and insulin. Appropriate anti-diabetic lifestyle changes are also well known in the art. Non-limiting examples include increased physical activity, caloric intake restriction, nutritional meal planning, and weight reduction. However, the invention is not so limited and other appropriate treatments will be apparent to one of ordinary skill in the art.

When a therapeutic agent (e.g., anti-diabetic compound) or other treatment is administered, it is administered in an amount effective to treat an existing glucose-related metabolic disorder or reduce the likelihood (or risk) of a future glucose-related metabolic disorder. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain lipids that are indicative of presence or risk a glucose-related metabolic disorder. It should be understood that the therapeutic agents of the invention are used to treat and/or prevent glucose-related metabolic disorders. Thus, in some cases, they may be used prophylactically in human subjects at risk of developing a glucose-related metabolic disorder. Thus, in some cases, an effective amount is that amount which can lower the risk of, slow or perhaps prevent altogether the development of a glucose-related metabolic disorder. It will be recognized when the therapeutic agent is used in acute circumstances, it is used to prevent one or more medically undesirable results that typically flow from such adverse events.

After one or more doses of a treatment have been administered, the present methods can be used to monitor efficacy, wherein a decrease in a level or ratio of a lipid associated with increased risk, or an increase in a level or ratio of a lipid associated with decreased risk, would indicate that the treatment is effective in reducing risk.

Methods for selecting a suitable treatment and an appropriate dose thereof will be apparent to one of ordinary skill in the art.

### EXAMPLES

The invention is further described in the following examples, which do not limit the scope of the invention described in the claims.

Statistical analyses for the following examples were performed as follows. Lipid levels were log-transformed, because raw data were highly skewed. Lipid levels, and the percent change in lipid levels with acute perturbation, were compared in the FHS matched-pair sample using paired t-tests. Conditional (matched pairs) logistic regression analyses were also performed to estimate the relative risk of diabetes at different lipid values. For these analyses, the lipid analytes were analyzed as continuous variables (log transformed and scaled to standard deviation [SD] of 1) and also as categorical variables (values 1, 2, 3, 4 were assigned using as cut-points the sex-specific quartiles of the lipids in controls). Regression analyses were adjusted for age, sex, BMI, fasting glucose, fasting insulin, total triglycerides, and HDL cholesterol. Case-control pairs were broken, however, for the comparison of cases in the bottom quartile of homeostatic model assessment of insulin resistance (HOMA-IR) versus controls in the bottom quartile of HOMA-IR. Spearman correlation coefficients were calculated between lipid levels and HOMA-IR. A p-value for trend was obtained by entering the quartile score into the model as variable, where the lowest quartile was considered the referent. All analyses were performed using SAS software version 9.1.3 (SAS Institute, Cary, N.C.).

### Example 1

### Establishing a Nested Case-Control Study to Enable Identification of Lipid Predictors of Type 2 Diabetes

The Offspring Cohort of the Framingham Heart Study (FHS) was initiated in 1971, when 5,124 individuals were enrolled into a longitudinal cohort study (8). Participants in this cohort are examined approximately every 4 years. The 5^{th} examination of this cohort took place in 1991 through 1995. Of 3,799 attendees at the 5^{th} examination (referred to as the baseline examination), 2,422 were eligible for the present investigation because they were free of diabetes (i.e., fasting glucose<126 mg/dl and not on glucose-lowering medications) and cardiovascular disease, were age 35 years or older, and underwent a standard 2-hour/75 g OGTT after a 12-hour overnight fast. Information on dietary intake was systematically obtained from a detailed, validated food frequency questionnaire (30). At each subsequent quadrennial visit, participants underwent a physician-administered physical examination and medical history, and routine laboratory tests. The presence of diabetes was ascertained at each visit, and defined by a fasting glucose≧126 mg/dl or the use of glucose-lowering medications including insulin (31). The homeostasis model assessment was used as a measure of relative insulin resistance as in Matthews et al (32).

Nested case-control design was as follows. During follow-up over 3 examinations (up to 12 years), 193 individuals developed new-onset type 2 diabetes in FHS. Logistic regression models were used to generate propensity scores for these 193 cases, using age, BMI, fasting glucose, and hypertension (defined as blood pressure≧140/90 mm Hg or use of anti-hypertensive therapy); a separate model was estimated for each follow-up examination of each sex. Each case was matched to the control with the closest exam- and sex-specific propensity score (within 0.10 on a scale of 0.0 to 1.0). A propensity-matched control was identified for all but 4 cases, yielding a final study sample of 189 cases and 189 controls.

Pharmacologic studies were performed as follows. Non-diabetic individuals >18 years of age were enrolled in the ongoing Study to Understand the Genetics of the Acute Response to Metformin and Glipizide in Humans (SUGAR MGH) at the Massachusetts General Hospital. At their first visit, participants received a single dose of glipizide 5 mg while fasting—plasma was collected and glucose and insulin levels were measured at time 0, 60 minutes, and 120 minutes. After a washout period of 6 days, subjects received metformin 500 mg twice daily for two days in order to reduce hepatic gluconeogenesis, and then underwent a 75 g OGTT in the presence of metformin. Post metformin samples at time 0 were compared to plasma drawn on the baseline visit prior to glipizide administration. From the first 164 subjects completing the protocol, 20 participants were selected who represented both high and low ends of the HOMA-IR range.

Between 1991-1995, designated as the “baseline” examination for the present investigation, 2,964 individuals from this cohort underwent OGTT. Since that time, a total of 193 individuals have developed new-onset type 2 diabetes over a 12-year follow up period. These individuals were designated as cases, and propensity score matching was used to select paired controls on the basis of age, sex, BMI, fasting glucose, and hypertension status. Using this approach, a matched control was identified for all but 4 cases, yielding a final study sample of 189 cases and 189 controls. Characteristics of the FHS study sample are shown in Table 1.

TABLE 1
Baseline characteristics of the FHS study sample

| | Individuals who | Individuals who |
|---|---|---|
| | developed diabetes | did not develop |
| | (n = 189) | diabetes (n = 189) |
| Clinical characteristics | | |
| Age, years | 56 ± 9 | 57 ± 8 |
| Women, % | 42% | 42% |
| BMI, kg/m² | 30.5 ± 5.0 | 30.0 ± 5.5 |
| Waist circumference, cm | 40.3 ± 4.8 | 39.2 ± 5.3 |
| Hypertension, % | 53% | 53% |
| Parental history of diabetes^{A}, % | 28% | 15% |
| Physical activity index | 35 ± 6.2 | 35 ± 7.3 |
| Other laboratory tests | | |
| Fasting glucose, mg/dl | 105 ± 9 | 105 ± 9 |
| 2-hour glucose (OGTT), mg/dl | 126 ± 32 | 118 ± 30 |
| Fasting insulin, uU/ml | 13.7 ± 9.9 | 11.9 ± 8.8 |
| HOMA-IR | 3.5 ± 2.6 | 3.1 ± 2.3 |
| Serum triglycerides^{B}, mg/dl | 192 ± 114 | 151 ± 90 |
| Total cholesterol, mg/dl | 212 ± 36 | 209 ± 36 |
| HDL cholesterol^{B}, mg/dl | 43 ± 12 | 47 ± 14 |
| Serum creatinine, mg/dl | 0.83 ± 0.24 | 0.88 ± 0.23 |

Values are mean ± SD, or percentage.
^{A}Parental history information missing in 57 participants.
^{B}p < 0.05 for difference between cases and controls.
HOMA-IR: homeostasis model assessment of insulin resistance.
OGTT: oral glucose tolerance test.

As expected, there were no statistically significant baseline differences between cases and controls with respect to variables incorporated into the matching process. However, there were significant differences in total triglycerides (p<0.0001) and HDL cholesterol (p=0.0007) between cases and controls, establishing a unique opportunity to explore the role of dyslipidemia in type 2 diabetes prediction.

### Example 2

### Lipid Profiling Identifies a Lipid Pattern of Diabetes Risk

Lipid profiling was performed on fasting pre- and 2-hour post-OGTT plasma samples obtained from the baseline examination for all 378 FHS study participants.

Lipid profiling. Plasma lipid profiles were obtained using a 4000 QTRAP triple quadrupole mass spectrometer (Applied Biosystems/Sciex), coupled to a 1200 Series pump (Agilent Technologies) and an HTS PAL autosampler (Leap Technologies). MultiQuant software (Version 1.1; Applied Biosystems/Sciex) was used for automated peak integration and peaks were manually reviewed for quality of integration. Ammonium acetate, acetic acid, and LC-MS grade solvents were purchased from Sigma-Aldrich. 10 μL of plasma were extracted with 190 μL of isopropanol containing an internal standard, 1-dodecanoyl-2-tridecanoyl-sn-glycero-3-phosphocholine (Avanti Polar Lipids). After centrifugation, supernatants were injected directly, followed by reverse phase chromatography using a 150×3.0 mm Prosphere HP C4 column (Grace); mobile phase A: 95:5:0.1 v/v/v 10 mM ammonium acetate/methanol/acetic acid; mobile phase B: 99.9:0.1 v/v methanol/acetic acid. The column was eluted isocratically with 80% mobile phase A for 2 minutes followed by a linear gradient to 20% mobile phase A over 1 minute, a linear gradient to 0% mobile phase A over 12 minutes, then 10 minutes at 0% mobile phase A. MS analyses were carried out using electrospray ionization and Q1 scans in the positive ion mode. Ion spray voltage was 5.0 kV, and source temperature was 400° C.

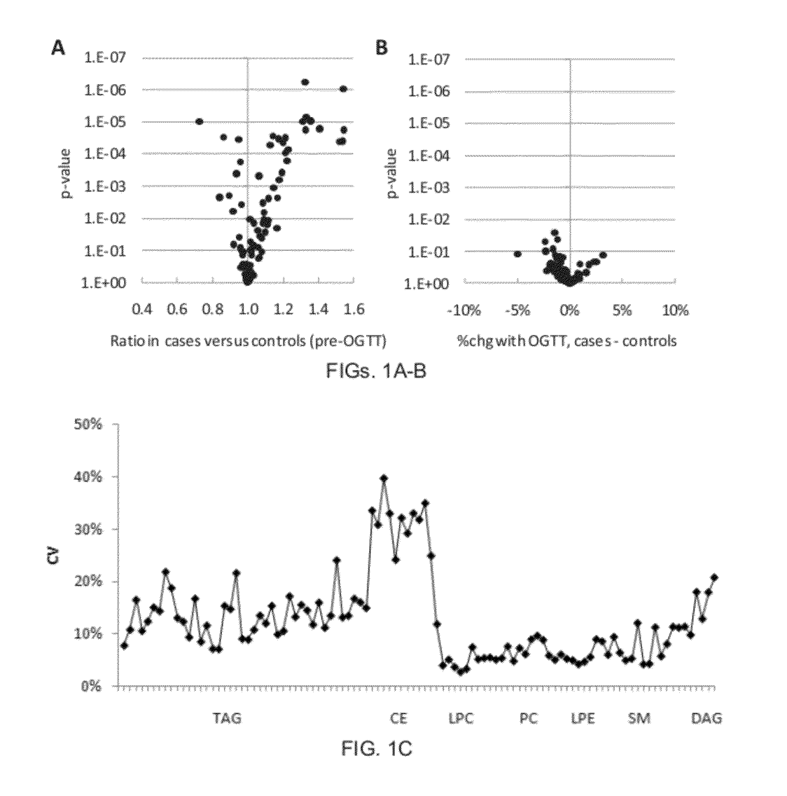

Internal standard peak areas were monitored for quality control and used to normalize analyte peak areas. In addition, replicates derived from a single pooled plasma sample were run after every 30 experimental samples, enabling detection of temporal drift in instrument performance. The CVs for each lipid analyte across a total of 29 pooled plasma samples are shown in FIG. 1C. Forty six percent of the analytes had CV≦10%, and 85% of the analytes had CV≦20%. For each lipid analyte, the first number denotes the total number of carbons in the lipid acyl chain(s) and the second number (after the colon) denotes the total number of double bonds in the lipid acyl chain(s).

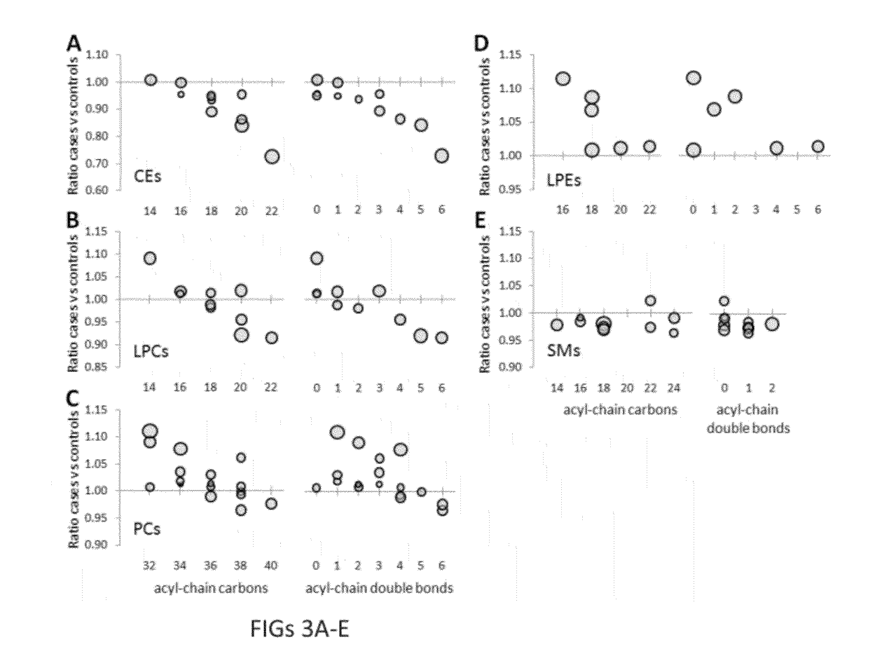

FIG. 1A shows the ratio of each lipid analyte in those who went on to develop diabetes (cases) versus those who did not (controls) in fasting pre-OGTT plasma. FIG. 1B shows the differences in OGTT-triggered lipid changes between cases and controls—as demonstrated in these figures, analyte levels in pre-OGTT plasma appeared to be more discriminating of case status than analyte responses to OGTT. While many lipids analytes were higher in cases than controls, some had the reverse association. The largest differences, regarding both the magnitude and significance of the association (as reflected by the p-value), were noted among TAGs. This result was not surprising given the imbalance in total triglycerides between cases and controls. However, a striking, downsloping pattern where TAGs of relatively lower carbon number and double bond content were most significantly elevated in cases relative to controls was also identified (FIG. 2A). When the comparison was restricted to the most insulin sensitive individuals, by focusing on the bottom quartiles of homeostasis model assessment of insulin resistance (HOMA-IR), the pattern was unchanged between cases versus controls (FIG. 2B)—mean HOMA-IR was 1.03 for cases and 1.01 for controls (p=0.36) in this subset. FIG. 3 shows that the downsloping relationship between diabetes risk and carbon number and double bond content was also present among CEs, LPCs, PCs and LPEs, but not for SMs.

### Example 3

### Diabetes Risk Pattern Persists after Adjustment in Multivariable Analysis

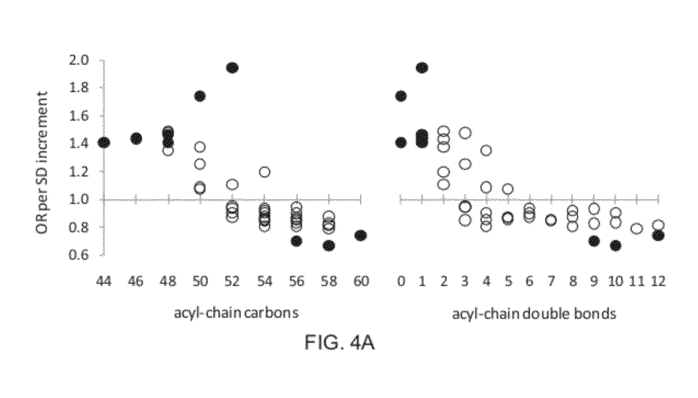

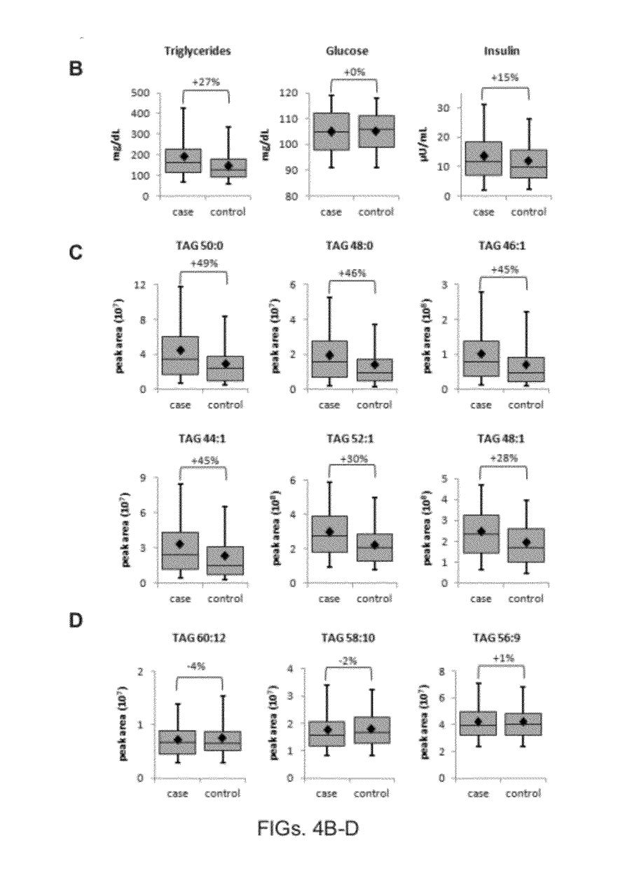

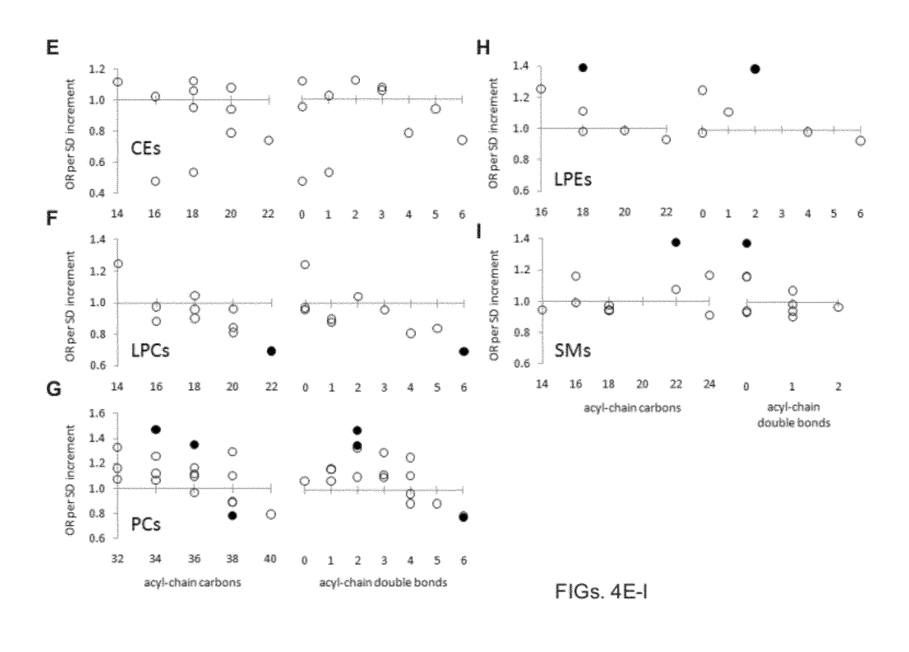

Given the imbalance in total triglycerides and HDL cholesterol between cases and controls at the baseline examination (Table 1), whether the relationship between diabetes risk and lipid carbon number and double bond content persisted after multivariable adjustment was tested. Conditional logistic regression models were fitted to assess the association between baseline lipid levels and future diabetes, adjusting for age, sex, BMI, fasting glucose, fasting insulin, total triglycerides, and HDL cholesterol. FIG. 4A depicts the odds ratio (OR) of diabetes per standard deviation (SD) increment in TAG level as a function of carbon number and double bond content, and shows that TAGs of lower carbon number and double bond content were associated with a OR>1 for diabetes, while TAGs of higher carbon number and double bond content were associated with a OR<1 for diabetes. The 9 TAGs that reached nominal significance (p<0.05) following multivariable adjustment, depicted as solid circles, are distributed at the extremes of saturation. Box and whisker plots for each of these TAGs in cases versus controls is depicted in FIGS. 4B-D. The heterogeneous correlation between these TAGs and total triglyceride measurements is shown in Table 2. The downsloping relationship between diabetes risk and carbon number and double bond content persisted after multivariable adjustment for LPCs, PCs, and possibly LPEs, but not for CEs (FIGS. 4E-I).

TABLE 2
TAG correlations with HOMA-IR and total triglycerides in FHS

| | Spearman | | Spearman | |
|---|---|---|---|---|
| | correlation with | | correlation with | |
| Lipid | HOMA-IR | P-value | total triglycerides | P-value |
| TAG 44:1 | 0.29 | <0.0001 | 0.63 | <0.0001 |
| TAG 46:2 | 0.33 | <0.0001 | 0.73 | <0.0001 |
| TAG 46:1 | 0.33 | <0.0001 | 0.73 | <0.0001 |
| TAG 48:4 | 0.26 | <0.0001 | 0.65 | <0.0001 |
| TAG 48:3 | 0.33 | <0.0001 | 0.85 | <0.0001 |
| TAG 48:2 | 0.35 | <0.0001 | 0.82 | <0.0001 |
| TAG 48:1 | 0.33 | <0.0001 | 0.66 | <0.0001 |
| TAG 48:0 | 0.35 | <0.0001 | 0.63 | <0.0001 |
| TAG 50:5 | 0.27 | <0.0001 | 0.68 | <0.0001 |
| TAG 50:4 | 0.33 | <0.0001 | 0.89 | <0.0001 |
| TAG 50:3 | 0.35 | <0.0001 | 0.90 | <0.0001 |
| TAG 50:2 | 0.33 | <0.0001 | 0.69 | <0.0001 |
| TAG 50:0 | 0.36 | <0.0001 | 0.73 | <0.0001 |
| TAG 52:6 | 0.24 | <0.0001 | 0.67 | <0.0001 |
| TAG 52:5 | 0.25 | <0.0001 | 0.74 | <0.0001 |
| TAG 52:4 | 0.21 | <0.0001 | 0.66 | <0.0001 |
| TAG 52:3 | 0.19 | 0.0002 | 0.51 | <0.0001 |
| TAG 52:2 | 0.29 | <0.0001 | 0.76 | <0.0001 |
| TAG 52:1 | 0.37 | <0.0001 | 0.84 | <0.0001 |
| TAG 54:10 | 0.26 | <0.0001 | 0.62 | <0.0001 |
| TAG 54:9 | 0.26 | <0.0001 | 0.72 | <0.0001 |
| TAG 54:8 | 0.15 | 0.0044 | 0.60 | <0.0001 |
| TAG 54:7 | 0.16 | 0.0015 | 0.57 | <0.0001 |
| TAG 54:6 | 0.17 | 0.0007 | 0.55 | <0.0001 |
| TAG 54:5 | 0.11 | 0.032 | 0.41 | <0.0001 |
| TAG 54:4 | 0.09 | 0.093 | 0.22 | <0.0001 |
| TAG 54:3 | 0.25 | <0.0001 | 0.66 | <0.0001 |
| TAG 54:2 | 0.33 | <0.0001 | 0.85 | <0.0001 |
| TAG 56:10 | 0.21 | <0.0001 | 0.61 | <0.0001 |
| TAG 56:9 | 0.11 | 0.027 | 0.50 | <0.0001 |
| TAG 56:8 | 0.11 | 0.041 | 0.41 | <0.0001 |
| TAG 56:7 | 0.03 | 0.62 | 0.32 | <0.0001 |
| TAG 56:6 | −0.07 | 0.2 | 0.10 | 0.045 |
| TAG 56:5 | 0.07 | 0.16 | 0.39 | <0.0001 |
| TAG 56:4 | 0.16 | 0.0021 | 0.56 | <0.0001 |
| TAG 56:3 | 0.27 | <0.0001 | 0.79 | <0.0001 |
| TAG 58:12 | 0.15 | 0.0029 | 0.46 | <0.0001 |
| TAG 58:11 | 0.12 | 0.019 | 0.44 | <0.0001 |
| TAG 58:10 | 0.10 | 0.058 | 0.40 | <0.0001 |
| TAG 58:9 | 0.01 | 0.78 | 0.22 | <0.0001 |
| TAG 58:8 | −0.07 | 0.17 | 0.04 | 0.48 |
| TAG 60:12 | 0.02 | 0.69 | 0.31 | <0.0001 |

HOMA-IR: homeostasis model assessment of insulin resistance

### Example 4

### Improving Diabetes Prediction Over Standard Clinical Measures

Following multivariable adjustment for age, sex, BMI, fasting glucose, fasting insulin, total triglycerides, and HDL cholesterol, a total of 15 lipid analytes (Table 3) reached nominal significance (p<0.05), including the 9 TAGs depicted in FIG. 4.

TABLE 3
Relationship of individual baseline lipid levels to risk of future diabetes

| | | | OR 1^{st} | OR 2^{nd} | OR 3^{rd} | OR 4^{th} | P-value |
|---|---|---|---|---|---|---|---|
| Lipid | OR per SD | P-value | quartile | quartile | quartile | quartile | for trend |
| TAG 52:1 | 1.94 (1.18-3.20) | 0.009 | 1.0 | 2.21 (1.01-4.83) | 1.74 (0.72-4.21) | 4.19 (1.39-12.62) | 0.032 |
| TAG 50:0 | 1.74 (1.19-2.57) | 0.005 | 1.0 | 2.02 (0.95-4.29) | 1.95 (0.87-4.37) | 3.86 (1.43-10.41) | 0.016 |
| PC 34:2 | 1.47 (1.06-2.04) | 0.021 | 1.0 | 2.12 (1.00-4.49) | 2.45 (1.07-5.58) | 2.89 (1.16-7.20) | 0.035 |
| TAG 48:1 | 1.47 (1.05-2.05) | 0.026 | 1.0 | 1.34 (0.63-2.84) | 1.32 (0.65-2.67) | 2.91 (1.23-6.91) | 0.023 |
| TAG 46:1 | 1.44 (1.01-2.06) | 0.043 | 1.0 | 1.10 (0.53-2.30) | 1.32 (0.63-2.76) | 2.23 (0.95-5.22) | 0.054 |
| TAG 48:0 | 1.41 (1.01-1.95) | 0.042 | 1.0 | 0.79 (0.39-1.59) | 1.04 (0.52-2.10) | 2.15 (0.96-4.78) | 0.051 |
| TAG 44:1 | 1.41 (1.02-1.94) | 0.036 | 1.0 | 0.94 (0.47-1.85) | 1.35 (0.66-2.77) | 1.61 (0.74-3.48) | 0.17 |
| LPE 18:2 | 1.39 (1.07-1.81) | 0.016 | 1.0 | 1.73 (0.86-3.51) | 1.86 (0.90-3.88) | 2.67 (1.30-5.46) | 0.001 |
| SM 22:0 | 1.38 (1.05-1.81) | 0.022 | 1.0 | 1.09 (0.54-2.20) | 1.62 (0.85-3.10) | 2.56 (1.18-5.56) | 0.015 |
| PC 36:2 | 1.35 (1.02-1.80) | 0.039 | 1.0 | 1.18 (0.61-2.30) | 1.72 (0.83-3.53) | 1.35 (0.61-2.99) | 0.35 |
| TAG 58:10 | 0.67 (0.50-0.89) | 0.006 | 1.0 | 0.56 (0.30-1.07) | 0.49 (0.26-0.95) | 0.30 (0.14-0.67) | 0.003 |
| LPC 22:6 | 0.69 (0.53-0.90) | 0.006 | 1.0 | 0.76 (0.42-1.36) | 0.57 (0.30-1.09) | 0.38 (0.18-0.79) | 0.008 |
| TAG 56:9 | 0.70 (0.52-0.94) | 0.017 | 1.0 | 0.89 (0.46-1.69) | 0.57 (0.29-1.10) | 0.46 (0.21-1.01) | 0.019 |
| TAG 60:12 | 0.74 (0.58-0.96) | 0.022 | 1.0 | 0.51 (0.27-0.97) | 0.74 (0.41-1.35) | 0.56 (0.28-1.11) | 0.17 |
| PC 38:6 | 0.78 (0.61-1.00) | 0.049 | 1.0 | 0.78 (0.43-1.40) | 0.63 (0.34-1.20) | 0.51 (0.26-1.00) | 0.041 |
| TAG 50:0 + TAG 58:10 | 2.72 (1.55-4.76) | 0.001 | 1.0 | 1.80 (0.88-3.69) | 3.25 (1.39-7.61) | 5.36 (1.94-14.80) | 0.001 |

Values are odds ratios (95% confidence intervals) for diabetes, from conditional logistic regressions. All models adjusted for age, sex, BMI, fasting glucose, fasting insulin, triglycerides, and HDL cholesterol. Analytes are ordered by OR per SD values. Trend test used integers for quartile values. Each individual was assigned to a quartile based on the cutpoint values calculated in the control sample.

These findings were largely unchanged when the model was further adjusted for parental history of diabetes (Table 4).

TABLE 4
Lipid levels and risk of future diabetes adjusted for parental history

| | | | Baseline Model* + | |
|---|---|---|---|---|
| | Baseline Model* | | Parental History | |
| Lipid | OR per SD | P-value | OR per SD | P-value |
| TAG 52:1 | 1.94 | 0.009 | 1.78 | 0.026 |
| TAG 50:0 | 1.74 | 0.005 | 1.66 | 0.010 |
| PC 34:2 | 1.47 | 0.021 | 1.50 | 0.019 |
| TAG 48:1 | 1.47 | 0.026 | 1.41 | 0.048 |
| TAG 46:1 | 1.44 | 0.043 | 1.36 | 0.097 |
| TAG 48:0 | 1.41 | 0.042 | 1.40 | 0.046 |
| TAG 44:1 | 1.41 | 0.036 | 1.32 | 0.092 |
| LPE 18:2 | 1.39 | 0.016 | 1.38 | 0.018 |
| SM 22:0 | 1.38 | 0.022 | 1.29 | 0.072 |
| PC 36:2 | 1.35 | 0.039 | 1.32 | 0.059 |
| TAG 58:10 | 0.67 | 0.006 | 0.68 | 0.010 |
| LPC 22:6 | 0.69 | 0.006 | 0.69 | 0.007 |
| TAG 56:9 | 0.70 | 0.017 | 0.71 | 0.024 |
| TAG 60:12 | 0.74 | 0.022 | 0.75 | 0.030 |
| PC 38:6 | 0.78 | 0.049 | 0.77 | 0.047 |
| TAG 50:0 + TAG 58:10 | 2.72 | 0.001 | 2.72 | 0.001 |

*Adjusted for age, sex, BMI, fasting glucose, fasting insulin, triglycerides, and HDL cholesterol

For the 10 lipids associated with increased diabetes risk, each SD increment in log marker was associated with a 1.35 to 1.94 increased odds of future diabetes. Individuals in the top quartile of these lipid analytes had a 1.35 to 4.19-fold odds of developing diabetes over the 12-year follow up period, compared to individuals in the bottom quartile of these lipids. For the 5 negative predictors, each SD increment in log marker was associated with a 0.67 to 0.78 decreased odds of future diabetes. Individuals in the lowest quartile of these lipid analytes had a 0.30 to 0.56-fold odds of developing diabetes over the 12-year follow up period, compared to individuals in the referent quartile of these lipids. The combination of the most significant positive and negative predictors, TAG 50:0 and TAG 58:10, was associated with a OR of 2.72 per SD increment in biomarker level. Individuals in the top quartile of this combination had a 5.36 fold risk of developing diabetes, compared to individuals in the lowest quartile (p=0.0006 for trend), in models adjusting for age, sex, BMI, fasting glucose, fasting insulin, total triglycerides, and HDL cholesterol.

### Example 5

### Lipid Profiling Demonstrates a Heterogeneous TAG Response to OGTT

To explore potential mechanisms for the differential risk attributable to distinct TAGs, the TAG response to OGTT was examined across all 378 FHS study participants. Much of the biochemical response to glucose ingestion can be attributed to an endogenous rise in insulin (9). Interestingly, TAGs of lower carbon number and double bond content decreased, and TAGs of relatively higher carbon number and double bond content increased, in response to OGTT (FIG. 5A). The fall in TAGs of lower carbon number and double bond content was more pronounced in individuals in the lowest quartile of HOMA-IR relative to individuals in the highest quartile of HOMA-IR (FIG. 5B).

### Example 6

### Lipid Profiling Demonstrates a Heterogeneous Relationship Between Plasma TAGs and Insulin Resistance

Given the heterogeneous dynamic response of different TAGs to stimulation of the insulin axis, the relationship between TAG levels in fasting pre-OGTT plasma and HOMA-IR in the FHS sample (cases and controls) was examined. Across the TAGs, the Spearman correlation coefficient between individual TAGs and HOMA-IR ranged from −0.07 to 0.37 (Table 2). There was a pattern in which TAGs of relatively lower carbon number and double bond content were significantly and positively correlated with HOMA-IR, and TAGs of higher carbon number and double bond content were not correlated with HOMA-IR (FIG. 5C). That is, TAGs that fell in response to insulin stimulation were elevated in the context of insulin resistance. The results were unchanged if HOMA-IR was replaced by fasting insulin. The risk of diabetes attributable to each TAG, as determined by conditional logistic regression, was related to the TAG's correlation with HOMA-IR (FIG. 5D).

By contrast, the diabetes risk attributable to SM 22:0 was not clearly related to its correlation with HOMA-IR (r=0.03), which is consistent with its ability to add risk information to select TAGs. Adding SM 22:0 to the two TAGs that were the most significantly positive and negative predictors (TAG 50:0 and TAG 58:10) further strengthened disease prediction, with a 8.12 fold risk of developing diabetes in the highest versus the lowest quartile of this multimarker (p<0.0001 for trend).

### Example 7

Pharmacological manipulation of insulin release highlights the role of insulin action on plasma TAGs. OGTT causes an acute rise in plasma glucose, which then triggers a rise in insulin. In order to formally exclude the possibility that TAGs respond differentially to the rise in glucose rather than insulin, lipid profiling was performed on plasma from 20 non-diabetic individuals (Table 3) before, 60 minutes after, and 120 minutes after oral ingestion of an insulin secretagogue (glipizide 5 mg).

Acute exercise testing was performed as follows. Outpatients referred to the MGH Exercise Laboratory for diagnostic treadmill testing (n=50) were recruited. In order to study the normal metabolic response to exercise, subjects were selected who met the following inclusion criteria: 1) normal exercise tolerance as defined by estimated peak VO₂ greater than 70% predicted; 2) evident maximum effort on the basis of heart rate response greater than 85% predicted in the absence of beta-blockade; and 3) pre-exercise fasting for at least 4 hours. Exclusion criteria included cessation of exercise by the test supervisor, reversible perfusion defects or electrocardiographic evidence of exercise-induced ischemia, mechanical limitation to exercise, or left ventricular ejection fraction less than 50%. The 10 individuals with type 2 diabetes all carried a diagnosis of type 2 diabetes in the electronic medical record and were also receiving at least one anti-diabetes medication.

TABLE 3
Baseline characteristics of pharmacologic
and acute exercise cohorts

| | Pharma- | Acute Exercise Testing | | |
|---|---|---|---|---|
| | cologic | | Type 2 | No |
| | studies | Combined | diabetes | diabetes |
| | (n = 20) | (n = 50) | (n = 10) | (n = 40) |
| Clinical | | | | |
| characteristics | | | | |
| Age, years | 55 ± 18 | 63 ± 11 | 64 ± 4 | 63 ± 12 |
| Women, % | 50% | 12% | 10% | 13% |
| BMI, kg/m² | 31.7 ± 7.5 | 29.1 ± 4.0 | 29.8 ± 4.3 | 28.9 ± 3.9 |
| Hypertension, | 20% | 76% | 100% | 70% |
| % | | | | |
| Other | | | | |
| laboratory | | | | |
| tests | | | | |
| Fasting | 95 ± 13 | 118 ± 37 | 170 ± 54 | 105 ± 14 |
| glucose, | | | | |
| mg/dl | | | | |
| Fasting | 9.6 ± 6.7 | 13.6 ± 16.4 | 22.2 ± 17.0 | 11.4 ± 15.7 |
| insulin, | | | | |
| uU/ml | | | | |
| HOMA-IR | 2.3 ± 1.8 | 4.5 ± 6.0 | N/A^{A} | 3.1 ± 4.6 |
| Serum | ND | 138 ± 111 | 125 ± 51 | 141 ± 122 |
| triglycerides, | | | | |
| mg/dl | | | | |
| Total | ND | 178 ± 51 | 163 ± 46 | 181 ± 52 |
| cholesterol, | | | | |
| mg/dl | | | | |
| HDL | ND | 53 ± 15 | 45 ± 16 | 55 ± 15 |
| cholesterol, | | | | |
| mg/dl | | | | |

Values are mean ± SD, or percentage. HOMA-IR: homeostasis model assessment of insulin resistance.
^{A}Interpretation of HOMA-IR limited in individuals with type 2 diabetes due to intake of anti-diabetes medications.

As expected, glipizide administration led to an increase in mean plasma insulin (9.6 μU/mL [baseline]→23.5 μU/mL [60 minutes, p=0.0009 versus baseline]→17.8 μU/mL [120 minutes, p=0.094 versus baseline]), and a decrease in mean plasma glucose (95 mg/dL [baseline]→79 mg/dL [60 minutes, p<0.0001 versus baseline]→65 mg/dL [120 minutes, p<0.0001 versus baseline). FIGS. 6A (60 minutes versus baseline) and 6B (120 minutes versus baseline) show that glipizide administration recapitulated the TAG response to OGTT, suggesting that insulin rather than glucose mediates the observed changes.

After a washout period of 6 days, these 20 individuals were then administered four doses of metformin (500 mg) over two days. Although chronic metformin use decreases insulin resistance, its acute effect is to decrease hepatic glucose output, and as a result, lower plasma insulin (10). Consistent with these effects, a decrease in mean plasma glucose (95 mg/dL→86 mg/dL, p=0.022) and insulin (9.6 μU/mL 6.8 μl/mL, p=0.00037) was noted following metformin intake. With this fall in plasma insulin, an increase in TAGs of lower carbon number and double bond content, and a decrease in TAGs of higher carbon number and double bond content were noted, i.e. the inverse response compared to OGTT and glipizide administration (FIG. 6C).

### Example 8

### The Diabetes Risk Pattern in Tags Persists in Established Disease and is Ameliorated by Acute Exercise

Lipid profiling was also performed on 50 individuals undergoing treadmill stress testing (Table 3), including 10 individuals with type 2 diabetes. In this cohort, participants with diabetes had similar total triglycerides (125 mg/dL versus 141 mg/dL, p=0.71) and BMI (29.8 versus 28.9, p=0.54) as compared to the 40 participants without diabetes. FIG. 6D depicts the ratio of TAGs in individuals with diabetes (n=10) versus non-diabetic individuals (n=40), and demonstrates the same downsloping pattern of TAGs identified in the pre-diabetic state. FIG. 6E shows the change in TAGs that occurred with exercise treadmill testing across all 50 individuals, demonstrating a similar pattern to OGTT and glipizide administration. Exercise is known to acutely improve insulin sensitivity at the tissue level (11, 12), as demonstrated by the fall in plasma insulin (13.6 μU/mL→9.7 μU/mL, p=0.049) in the face of constant glycemia (118 mg/dL→118 mg/dL, p=0.76) with exercise in this cohort.

### Example 9

### Tandem Mass Spectrometry Identifies the Acyl Chain Constituents of Diabetes Predictors

Operating the mass spectrometer in “full scan” mode, the lipid profiling platform described herein distinguishes analytes on the basis of total acyl chain carbon number and double bond content. To unambiguously characterize the fatty acid constituents of TAGs, PCs, and DAGs (the molecular weight of each LPC, LPE, CE, and SM analyte identifies a specific acyl chain length and saturation, and so can be characterized using full scan MS), additional plasma MS-MS analyses were performed to systematically fragment each TAG, PC, and DAG in order to identify each analyte's acyl chain composition.

Tandem MS/MS analyses were performed as follows. MS/MS analyses of pooled plasma were obtained on a 4000 QTRAP triple quadrupole mass spectrometer. Sample extraction and chromatography were performed as above. Following electrospray ionization, enhanced product ion scans were performed in the positive ion mode for each TAG, PC, and DAG monitored by the lipid profiling platform, as well as for LPC 22:6, SM 22:0, and LPE 18:2. The Na⁺ adduct of each TAG was fragmented, and product ion scans were analyzed for the neutral loss of individual acyl chains as either a R—COOH or R—COONa molecule. The H⁺ adduct of each PC was also fragmented, and product ion scans were monitored for the neutral loss of acyl chains as a R—COOH molecule and for the neutral loss of phosphocholine. Product ion scans for LPC 22:6, SM 22:0, and LPE 18:2 were monitored for the neutral loss of phosphocholine (LPC 22:6 and SM 22:0) or phosphoethanolamine (LPE 18:2). Ion spray voltage was 5.0 kV, source temperature was 450° C., and collision energies were set between 33 and 70.

FIG. 7 depicts the identified acyl chain constituents of lipid analytes that predict diabetes in FHS following multivariable adjustment. Lipids associated with OR>1 for diabetes are primarily comprised of saturated or monounsaturated fatty acids whereas lipids associated with OR<1 for diabetes are primarily comprised of polyunsaturated fatty acids.

### Example 10

### Dietary Intake does not Explain the Diabetes Risk Lipid Pattern

Because dietary habits among FHS participants are captured through administration of a food frequency questionnaire, whether the diabetes risk pattern observed was attributable to dietary differences was tested. No correlation was found between the percent of total fat intake from saturated fats (37.4% cases versus 38.0% controls, p=0.15) or polyunsaturated fats (21.4% cases versus 21.4% controls, p=0.81) and case status. There was a trend for higher overall saturated fat intake (23.4 g versus 21.4 g, p=0.054), and significantly higher polyunsaturated fat intake (13.3 g versus 11.8 g, p=0.011) among cases versus controls.

### REFERENCES

1. Chien, K., Cai, T., Hsu, H., Su, T., Chang, W., Chen, M., Lee, Y., and Hu, F. B. 2009. A prediction model for type 2 diabetes risk among Chinese people. Diabetologia 52:443-450.
2. D'Agostino, R. B., Jr., Hamman, R. F., Karter, A. J., Mykkanen, L., Wagenknecht, L. E., and Haffner, S. M. 2004. Cardiovascular disease risk factors predict the development of type 2 diabetes: the insulin resistance atherosclerosis study. Diabetes Care 27:2234-2240.
3. Kahn, H. S., Cheng, Y. J., Thompson, T. J., Imperatore, G., and Gregg, E. W. 2009. Two risk-scoring systems for predicting incident diabetes mellitus in U.S. adults age 45 to 64 years. Ann Intern Med 150:741-751.
4. Schulze, M. B., Weikert, C., Pischon, T., Bergmann, M. M., Al-Hasani, H., Schleicher, E., Fritsche, A., Haring, H. U., Boeing, H., and Joost, H. G. 2009. Use of multiple metabolic and genetic markers to improve the prediction of type 2 diabetes: the EPIC-Potsdam Study. Diabetes Care 32:2116-2119.
5. Wilson, P. W., Meigs, J. B., Sullivan, L., Fox, C. S., Nathan, D. M., and D'Agostino, R. B., Sr. 2007. Prediction of incident diabetes mellitus in middle-aged adults: the Framingham Offspring Study. Arch Intern Med 167:1068-1074.
6. Stein, E. A., and Myers, G. L. 1995. National Cholesterol Education Program recommendations for triglyceride measurement: executive summary. The National Cholesterol Education Program Working Group on Lipoprotein Measurement. Clin Chem 41:1421-1426.
7. Wilson, P. W., D'Agostino, R. B., Levy, D., Belanger, A. M., Silbershatz, H., and Kannel, W. B. 1998. Prediction of coronary heart disease using risk factor categories. Circulation 97:1837-1847.
8. Kannel, W. B., Feinleib, M., McNamara, P. M., Garrison, R. J., and Castelli, W. P. 1979. An investigation of coronary heart disease in families: the Framingham Offspring Study. Am J Epidemiol 110:281-290.
9. Shaham, O., Wei, R., Wang, T. J., Ricciardi, C., Lewis, G. D., Vasan, R. S., Can, S. A., Thadhani, R., Gerszten, R. E., and Mootha, V. K. 2008. Metabolic profiling of the human response to a glucose challenge reveals distinct axes of insulin sensitivity. Mol Syst Biol 4:214.
10. Chu, C. A., Wiernsperger, N., Muscato, N., Knauf, M., Neal, D. W., and Chemington, A. D. 2000. The acute effect of metformin on glucose production in the conscious dog is primarily attributable to inhibition of glycogenolysis. Metabolism 49:1619-1626.
11. Henriksen, E. J. 2002. Invited review: Effects of acute exercise and exercise training on insulin resistance. J Appl Physiol 93:788-796.
12. Holloszy, J. O. 2005. Exercise-induced increase in muscle insulin sensitivity. J Appl Physiol 99:338-343.
13. German, J. B., Gillies, L. A., Smilowitz, J. T., Zivkovic, A. M., and Watkins, S. M. 2007. Lipidomics and lipid profiling in metabolomics. Curr Opin Lipidol 18:66-71.
14. Seppanen-Laakso, T., and Oresic, M. 2009. How to study lipidomes. J Mol Endocrinol 42:185-190.
15. Wenk, M. R. 2005. The emerging field of lipidomics. Nat Rev Drug Discov 4:594-610.
16. Wopereis, S., Rubingh, C. M., van Erk, M. J., Verheij, E. R., van Vliet, T., Cnubben, N. H., Smilde, A. K., van der Greef, J., van Ommen, B., and Hendriks, H. F. 2009. Metabolic profiling of the response to an oral glucose tolerance test detects subtle metabolic changes. PLoS One 4: e4525.
17. Zhao, X., Peter, A., Fritsche, J., Elcnerova, M., Fritsche, A., Haring, H. U., Schleicher, E. D., Xu, G., and Lehmann, R. 2009. Changes of the plasma metabolome during an oral glucose tolerance test: is there more than glucose to look at? Am J Physiol Endocrinol Metab 296:E384-393.
18. Schwab, U., Seppanen-Laakso, T., Yetukuri, L., Agren, J., Kolehmainen, M., Laaksonen, D. E., Ruskeepaa, A. L., Gylling, H., Uusitupa, M., and Oresic, M. 2008. Triacylglycerol fatty acid composition in diet-induced weight loss in subjects with abnormal glucose metabolism—the GENOBIN study. PLoS One 3:e2630.
19. Lyssenko, V., Jonsson, A., Almgren, P., Pulizzi, N., Isomaa, B., Tuomi, T., Berglund, G., Altshuler, D., Nilsson, P., and Groop, L. 2008. Clinical risk factors, DNA variants, and the development of type 2 diabetes. N Engl J Med 359:2220-2232.
20. Hodge, A. M., English, D. R., O'Dea, K., Sinclair, A. J., Makrides, M., Gibson, R. A., and Giles, G. G. 2007. Plasma phospholipid and dietary fatty acids as predictors of type 2 diabetes: interpreting the role of linoleic acid. Am J Clin Nutr 86:189-197.
21. Laaksonen, D. E., Lakka, T. A., Lakka, H. M., Nyyssonen, K., Rissanen, T., Niskanen, L. K., and Salonen, J. T. 2002. Serum fatty acid composition predicts development of impaired fasting glycaemia and diabetes in middle-aged men. Diabet Med 19:456-464.
22. Vessby, B., Aro, A., Skarfors, E., Berglund, L., Salminen, I., and Lithell, H.1994. The risk to develop NIDDM is related to the fatty acid composition of the serum cholesterol esters. Diabetes 43:1353-1357.
23. Wang, L., Folsom, A. R., Zheng, Z. J., Pankow, J. S., and Eckfeldt, J. H. 2003. Plasma fatty acid composition and incidence of diabetes in middle-aged adults: the Atherosclerosis Risk in Communities (ARIC) Study. Am J Clin Nutr 78:91-98.
24. Parks, E. J., and Hellerstein, M. K. 2006. Thematic review series: patient-oriented research. Recent advances in liver triacylglycerol and fatty acid metabolism using stable isotope labeling techniques. J Lipid Res 47:1651-1660.
25. Guillou, H., Zadravec, D., Martin, P. G., and Jacobsson, A. The key roles of elongases and desaturases in mammalian fatty acid metabolism: Insights from transgenic mice. Prog Lipid Res 49:186-199.
26. Nakamura, M. T., and Nara, T. Y. 2004. Structure, function, and dietary regulation of delta6, delta5, and delta9 desaturases. Annu Rev Nutr 24:345-376.
27. Brenner, R. R. 2003. Hormonal modulation of delta6 and delta5 desaturases: case of diabetes. Prostaglandins Leukot Essent Fatty Acids 68:151-162.
28. Nakamura, M. T., and Nara, T. Y. 2002. Gene regulation of mammalian desaturases. Biochem Soc Trans 30:1076-1079.
29. Kotronen, A., Velagapudi, V. R., Yetukuri, L., Westerbacka, J., Bergholm, R., Ekroos, K., Makkonen, J., Taskinen, M. R., Oresic, M., and Yki-Jarvinen, H.2009. Serum saturated fatty acids containing triacylglycerols are better markers of insulin resistance than total serum triacylglycerol concentrations. Diabetologia 52:684-690.
30. Rimm, E. B., Giovannucci, E. L., Stampfer, M. J., Colditz, G. A., Litin, L. B., and Willett, W. C. 1992. Reproducibility and validity of an expanded self-administered semiquantitative food frequency questionnaire among male health professionals. Am. J. Epidemiol. 135:1114-1126.
31. 1997. Report of the Expert Committee on the Diagnosis and Classification of Diabetes Mellitus. Diabetes Care 20:1183-1197.
32. Matthews, D. R., Hosker, J. P., Rudenski, A. S., Naylor, B. A., Treacher, D. F., and Turner, R. C. 1985. Homeostasis model assessment: insulin resistance and beta-cell function from fasting plasma glucose and insulin concentrations in man. Diabetologia 28:412-419.
33. Suhre et al., Metabolic Footprint of Diabetes: A Multiplatform Metabolomics Study in an Epidemiological Setting. PLoS ONE 5(11): e13953 (2010).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the risk of developing diabetes in a subject, the method comprising:
determining levels of one or more lipids in the sample, wherein the lipids are selected from the group consisting of TAG 52:1, TAG 50:0, PC 34:2, TAG 48:1, TAG 46:1, TAG 48:0, TAG 44:1, LPE 18:2, SM 22:0, and PC 36:2;
wherein the presence of levels of TAG 52:1, TAG 50:0, PC 34:2, TAG 48:1, TAG 46:1, TAG 48:0, TAG 44:1, LPE 18:2, SM 22:0, or PC 36:2 above a threshold level indicates an increased risk of developing diabetes in the subject.

2. The method of claim 1, comprising determining levels of TAG 50:0.

3. The method of claim 1, comprising determining levels of TAG 50:0 and SM 22:0.

4. The method of claim 1, wherein the subject has normal glucose tolerance.

5. The method of claim 1, wherein the sample comprises serum or plasma from the subject.

6. The method of claim 1, further comprising selecting a treatment for the subject based on the lipids present in the sample.

7. The method of claim 6, further comprising administering the selected treatment to the subject.

8. The method of claim 6, wherein the treatment is administering to the subject an effective amount of at least one anti-diabetes compound.

9. The method of claim 1, wherein the subject has at least one risk factor for diabetes.

10. The method of claim 1, wherein the levels of the lipids are determined using a mass spectrometer.

11. A method for determining the risk of developing diabetes in a subject, the method comprising:
determining levels of one or more lipids in the sample, wherein the lipids are selected from the group consisting of TAG 58:10, LPC 22:6, TAG 56:9, TAG 60:12, and PC 38:6; and
wherein the presence of levels of TAG 58:10, LPC 22:6, TAG 56:9, TAG 60:12, or PC 38:6 above a threshold level indicates a decreased risk of developing diabetes in the subject.

12. The method of claim 11, comprising determining levels of TAG 58:10.

13. The method of claim 11, wherein the subject has normal glucose tolerance.

14. The method of claim 11, wherein the sample comprises serum or plasma from the subject.

15. The method of claim 11, further comprising selecting a treatment for the subject based on the lipids present in the sample.

16. The method of claim 15, further comprising administering the selected treatment to the subject.

17. The method of claim 15, wherein the treatment is administering to the subject an effective amount of at least one anti-diabetes compound.

18. The method of claim 11, wherein the subject has at least one risk factor for diabetes.

19. The method of claim 11, wherein the levels of the lipids are determined using a mass spectrometer.

20. A kit for determining the presence or risk of a glucose related metabolic disorder in a subject, the kit comprising:
one or more control samples comprising predetermined levels TAG 52:1, TAG 50:0, PC 34:2, TAG 48:1, TAG 46:1, TAG 48:0, TAG 44:1, LPE 18:2, SM 22:0, PC 36:2, TAG 58:10, LPC 22:6, TAG 56:9, TAG 60:12, and PC 38:6; and
instructions for use of the kit for determining the presence or risk of a glucose related metabolic disorder.
